(19) United States
(12) Patent Application Publication
(10) Pub. No.: US 2010/0317076 A1
(43) Pub. Date: Dec. 16, 2010

(54) STRAIN OF YARROWIA LIPOLYTICA AND ITS USE IN THE INDUSTRIAL RECLAMATION OF GLYCEROL FRACTIONS OBTAINED DURING BIODIESEL PRODUCTION

(76) Inventor: Waldemar Rymowicz, Wroclaw (PL)

Correspondence Address: COOPER & DUNHAM, LLP, 30 Rockefeller Plaza, 20th Floor, NEW YORK, NY, 10112, US

(21) Appl. No.: 12/741,311

(22) PCT Filed: Nov. 5, 2008
(86) PCT No.: PCT/PL08/00077

§ 371 Date: Aug. 31, 2010

(30) Foreign Application Priority Data
Nov. 5, 2007 (PL) P383685

(51) Int. Cl. C12P 7/20; C12N 1/16

(52) U.S. Cl. 435/159; 435/255.1

(57) ABSTRACT

An industrial method of reclaiming the glycerol fraction resulting from biodiesel production, a new strain of Yarrowia lipolytica for use in said method, and its use.

The subject of the present invention is an industrial method of reprocessing the glycerol fraction obtained during the production of biodiesel as well as a novel strain of Yarrowia lipolytica particularly suited for use in this process.

The production of reusable fuel oil components, the so called biodiesels, chiefly consists of producing fatty acid esters from natural triglycerides (usu. plant lipids) via transestrification. U.S. Pat. No. 2,271,619 reveals a method of transforming glycerides of higher fatty acids into esters of short alcohols through the addition of a saturated monohydroxyl aliphatic alcohol of less than five carbons in the presence of an essentially anhydrous alkali metal hydroxide as a catalyst. According to said patent the process should take place in a reactor at a temperature of 86 to 212° F. (30 to 100° C.). The amount of alcohol should not exceed 1.75 equivalents of glyceride, and the catalyst should be from 0.1 to 0.5% by mass of glyceride.

Subsequent patents amend or supplement this patent. U.S. Pat. Nos. 2,360,844; 2,383,632; 2,383,580; 2,383,581; 2,383,614; 2,383,633; 2,383,596; and 2,383,599 respectively describe the consecutive variants of the method described in U.S. Pat. No. 2,271,619 consisting of: a) addition of the acid into the process and spray-drying; b) addition of a distillation step of the unreacted alcohol; c) evaluation of the catalyst activity, suggested pH of 5 to 7; d) application of a partial fatty acid ester technique; e) reclamation of unreacted alcohol and acidification of the liquid in order to improve the separation of esters and glycerol; f) transformation, using various methods, of partially reacted glycerides; g) supplementation of the monohydroxyl aliphatic alcohol (not methanol) with a portion of methanol in order to improve the separation of liquid phases; as well as h) addition of a solvent in order to improve phase separation.

Other patents propose more extended modifications and improvements. U.S. Pat. Nos. 2,494,366; 2,383,601; 3,963,699; 4,303,590; 4,371,470; 4,668,439; 5,399,731; 5,434,279; and 5,525,126 also largely build on the technology described in U.S. Pat. No. 2,271,619. They respectively relate to: a) addition of an appropriate amount of acidic catalyst to the alkaline catalyst; b) further addition acidic estrification catalyst; c) conducting the process under constant temperature and pressure conditions, from vacuum to atmospheric pressure, d) addition of a second alkaline catalysis stage; e) addition of a second estrification stage and removal of the alkyl ester with an absorbent; f) introduction of an alcohol in vapour form; g) conducting the reaction at a lower temperature with an increased amount of acid; g) introduction of an improved method of phase separation using acid; as well as h) use of a catalyst being a mixture of calcium acetate and barium acetate.

Regardless of the technology used, the chief by-product formed through the transestrification of triglycerides in need of reclamation is the so-called glyceride fraction consisting of soluble, hydrophylic products of the reaction, meaning glycerol, and residues of the unused catalyst, fatty acid esters and other reagents used in further stages of separation of the transestrification process, such as phosphoric acid and inorganic salts.

Reclamation and/or recycling of the glycerol fraction constitute a significant problem in the production of biodiesel.

The goal of the present invention is to deliver a method of easily reclaiming the glycerine fraction arising during biodiesel production. A particular goal of the present invention is to deliver an efficient method of industrially transforming the glycerol fraction into easily absorbed biomass. This method could be used on an industrial scale to reclaim glycerol fractions, taking into account the varying composition of said fraction depending on the biodiesel manufacturing process used. The biomass produced should be characterised by a high content of easily absorbable protein, vitamins and should be capable of being used as a feed additive.

Unexpectedly, such defined problems have been solved in the present invention.

The goal of the present invention is an industrial method of utilizing a glycerol fraction resulting from biodiesel characterised in that the yeast Yarrowia lipolytica is cultured on a medium consisting of an aqueous solution of 20.0 to 70.0 g/l of the glycerol fraction, from 8.5 to 15.0 g/l (NH₄)₂SO_{4.} from 1.5 to 6.5 g/l urea, from 0.5 to 3.0 g/l MgSO₄×7H₂O, from 0.1 to 2.0 g/l KH₂PO_{4.} from 0.1 to 2.0 g/l yeast extract, at a temperature of 25-35° C., and aeration at 0.2-4 L air/1 L medium/minute maintaining a pH between 2.5 and 7.5. to a substantial exhaustion of the glycerol in the medium, where the culture is maintained in a periodic manner, replacing a portion of the post-culture broth at the end of each cycle with fresh medium.

The method according to the present invention is meant for use on an industrial scale, wherein, preferentially, the culture is maintained in a volume of at least 1000 litres. Equally preferentially, a separated post-culture broth contains at from 15 to 35 g/L of yeast dry mass, whereas the production of dry mass occurs at a rate from 1.5 to 3.0 g/Lh, and the biomass production occurs at maximum efficiency from 0.4 to 0.5 g dry biomass/g glycerol fraction in the medium. Preferentially, the protein content of the dry biomass is from 30 to 50% by mass. Preferentially, the culture makes use of the Yarrowia lipolytica SKOTAN strain deposited in the IBPRS under the accession number KKP 2018 p.

The SKOTAN strain of Yarrowia lipolytica has been deposited at a bank acting in accordance with the treaty of Budapest, and maintained by the Institute of Biotechnology of the Food and Agriculture Industry (henceforth IBPRS), ul. Rakowiecka 36. 02-532 Warsaw and was given the accession number KKP 2018 p. This is a wild-type strain, selected from among many other strains of this species tested in the course of designing the present invention, belonging to the collection of the Wroclaw University of Environmental and Life Sciences. The selection criteria consisted mainly of culture conditions based on the glycerol fraction. First of all, this strain, out of all of the strains of Yarrowia lipolytica tested, yielded an exceptionally preferable efficiency of biomass production and a great tolerance for deleterious culture conditions such as increasing osmotic pressure and the relatively low pH of the medium. Due to this, culturing such a strain is much simpler, since the danger of its contamination by other microorganisms is much lower. At the same time, the biomass produced possesses preferable nutritional properties such as a high content of easily absorbable protein and vitamins, particularly vitamin B. Due to this, it may be used as a high-quality feed additive.

The next subject of the present invention is the SKOTAN strain of Yarrowia lipolytica SKOTAN deposited in the IBPRS under the accession number KKP 2018 p.

The subject of the present invention is also the use of the SKOTAN strain of Yarrowia lipolytica SKOTAN deposited in the IBPRS under the accession number KKP 2018 p in the reclamation of waste arising from biodiesel production. Preferentially, the biomass produced is used in the manufacture of feeds.

### EXAMPLE 1

The composition of a medium (Medium 1) in the production of biomass of the yeast Yarrowia lipolytica on a medium based on a glycerol fraction from biodiesel production consists of (in g/litre):

| | |
|---|---|
| glycerol fraction | 20.0 to 70.0. preferentially about 50.0 |
| (NH₄)₂SO₄ | 8.5-15.0. preferentially about 12.6 |
| Urea | 1.5-6.5. preferentially about 4.0 |
| MgSO₄ × 7H₂O | 0.5-3.0. preferentially about 1.0 |
| KH₂PO₄ | 0.1-2.0. preferentially about 0.5 |
| Yeast extract | 0.1-2.0. preferentially about 0.5 |
| Tap water | to 1000 ml |
| pH | 2.5-7.5. preferentially about 3.5-4.0 |

In the proportions outlined above, the components of Medium 1 should be weighed out to 1100 L and brought to 1000 L with tap water.

Following the complete dissolution of the medium components, they are transferred into a bioreactor. The medium should be supplemented with 100 L of yeast cells pre-cultured in a bioreactor with a working volume of 150 L, cultured in a medium as above.

Culture conditions for various strains of Yarrowia lipolytica are:

a temperature of 25-35° C. (preferentially about 30° C.±1), with agitation at 400-1200 RPM (preferentially about 700-800), aeration at 0.2-4 L air/L medium/minute (preferentially about 1-1.5 L air/L medium/minute). The pH should be automatically maintained using 10 N NaOH. Should excessive foaming occur, a defoaming agent such as ACEPOL or another should be used.

The culture should be maintained until exhaustion of glycerol in the culture medium. 200 L of the cell suspension should be retained, into which the components of (Medium 1) should be added at a mass sufficient for 1100 L, and 900 L water should be added. This culture method (periodic, cyclical) may be repeated from 5 to 15 times.

This yeast propagation protocol yields 15-35 g/L (preferentially about 33 g/L), of dry yeast mass at a rate of 1.5-3.0 g/Lh (preferentially about 2.5 g/Lh), at a n efficiency of at least 0.4-0.5 g dry yeast mass/g glycerol fraction, (preferentially about 0.60 g/g in the case of SKOTAN Yarrowia lipolytica).

The protein content of the dry yeast should be 30-50%, (42% in the case of SKOTAN Yarrowia lipolytica).

The biomass obtained can be further processed, such as drying with well known techniques, particularly spray-drying, and then portioned and sold for use as a high-quality feed additive, particularly for bovine and poultry feeds.

1. An industrial method of reclaiming the glycerol fraction resulting from biodiesel production, characterised in that yeast of the species Yarrowia lipolytica is cultured on a medium consisting of an aqueous solution containing from 20.0 to 70.0 g/l of the glycerol fraction, from 8.5 to 15.0 g/l (NH₄)₂SO₄, from 1.5 to 6.5 g/l urea, from 0.5 to 3.0 g/l MgSO₄×7H₂O, from 0.1 to 2.0 g/l KH₂PO₄, from 0.1 to 2.0 g/l of yeast extract, at a temperature of 25-35° C., aeration at 0.2-4 L air/1L medium/minute, pH maintained at 2.5 to 7.5; up to the substantial exhaustion of glycerol in the medium, where the culture is maintained in a periodic fashion, replacing a portion of the post-culture broth with a fresh portion of medium following each cycle.

2. A method according to claim 1, characterised in that the culture is maintained in a volume of at least 1000 litres.

3. A method according to claim 1, characterised in that the separated post-culture broth yields from 15 to 35 g/L of yeast dry mass.

4. A method according to claim 1, characterised in that the biomass production is performed at a rate from 1.5 to 3.0 g/Lh.

5. A method according to claim 1, characterised in that the biomass production occurs with an overall efficiency of 0.4 to 0.5 g dry biomass/g glycerol fraction in the medium.

6. A method according to claim 1, characterised in that the dry biomass protein content is 30 to 50% by mass.

7. A method according to claim 1, characterised in that w the culture makes use of the SKOTAN strain of Yarrowia lipolytica, deposited in the IBPRS under the accession number KKP 2018 p.

8. The SKOTAN strain of Yarrowia lipolytica deposited in the IBPRS under the accession number KKP 2018 p.

9. The use of the SKOTAN strain of Yarrowia lipolytica deposited in the IBPRS under the accession number KKP 2018 p in the reclamation of waste products of biodiesel production.

10. A use according to claim 9, characterised in that the biomass produced is used in feed production.
